(19) United States
(12) Patent Application Publication
(10) Pub. No.: US 2026/0209210 A1
(43) Pub. Date: Jul. 23, 2026

(54) NEW SUBSTITUTED INDOLE-2-CARBOXAMIDES AS PHGDH INHIBITORS

(71) Applicant: Boehringer Ingelheim International GmbH, Ingelheim am Rhein (DE)

(72) Inventors: Matthias TREU, Vienna (AT); Harald WIENSTABL, Wien (AT)

(21) Appl. No.: 19/138,697

(22) PCT Filed: Dec. 12, 2023
(86) PCT No.: PCT/EP2023/085281

§ 371 Date: Jun. 13, 2025

(30) Foreign Application Priority Data
Dec. 15, 2022 (EP) 22213829.9

(51) Int. Cl. C07D 405/12; A61K 31/404

(52) U.S. Cl. CPC C07D 405/12; A61K 31/404

(57) ABSTRACT

The present invention encompasses compounds of formula (I) wherein the groups R1 to R8 have the meanings given in the claims and specification, their use as inhibitors of PHGDH, pharmaceutical compositions comprising such compounds and their use as medicaments, especially as agents for treatment and/or prevention of oncological diseases.

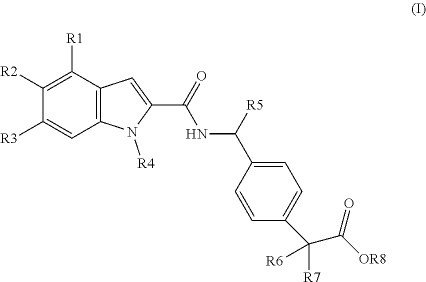

### FIELD OF THE INVENTION

The present invention relates to new indole-2-carboxamides of formula (I):

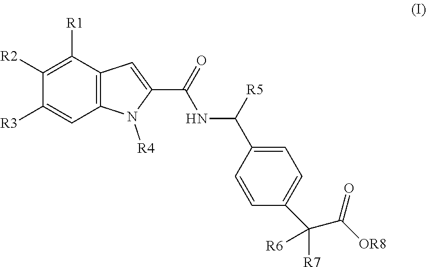

wherein the groups R1 to R8 have the meanings given in the claims and specification, their use as inhibitors of PHGDH, pharmaceutical compositions comprising such derivatives and their use as medicaments, especially as agents for treatment and/or prevention of oncological diseases.

### BACKGROUND OF THE INVENTION

The essential contribution of the serine synthetic pathway (SSP) to tumorigenesis has been shown by a plethora of studies. Serine (Ser) is a non-essential amino acid, which is indispensable for several cellular processes that are of particular importance for tumor cells: (i) Ser can be converted to glycine via the action of the serine hydroxymethyltransferase (SHMT) providing carbon units for purine nucleotide synthesis (Kalhan & Hanson, J Biol Chem. (2012) 287:19786-19791; Locasale, Nat Rev Cancer. (2013) 13:572-583; Amelio et al., Trends Biochem Sci. (2014) 39:191-198; Mehrmohamadi & Locasale Mol Cell Oncol. (2015) 2: e996418; Tedeschi et al., Cell Death Dis. (2013) 4: e877). (ii) Ser can react with palmitoyl-CoA to provide sphingosine required for the generation of sphingolipids that constitute the cell membrane (Ravez et al., J. Med. Chem. (2017) 60, 4:1227-1237; Xu et al., J Biol Chem. (1991) 266:2143-2150). (iii) Ser serves as a precursor of several amino acids like glycine and cysteine (Vazquez et al., Cancer Res. (2013) 73:478-482; Ravez et al., J. Med. Chem. (2017) 60, 4:1227-1237). (iv) Ser plays a crucial role in the regulation of the redox status due to the fact that serine is involved in the production of NADPH (Tedeschi et al., Cell Death Dis. (2013) 4: e877). (v) Last but not least, PHGDH, the key enzyme of the de novo SSP was shown to produce the oncometabolite D-2-hydroxyglutarate (D-2HG) which has been linked with epigenetic de-regulation in tumor cells (Mondesir et al., J Blood Med. (2016) 7:171-180; Fan et al., ACS Chem Biol. (2015) 10:510-516). The SSP not only provides essential building blocks/metabolites but also epigenetic regulators, Ser and its synthesis pathway essentially contribute to cell proliferation, tumor homeostasis and to de-differentiation of cancer cells (Mattaini et al., J Cell Biol. (2016) 214:249-257; El-Hattab, Mol Genet Metab. (2016) 118:153-159).

De novo synthesis of Ser is triggered via the SSP. The SSP diverts of the 3-PG from glycolysis to generate Ser as well as equimolar amounts of reduced nicotinamide adenine dinucleotide (NADH) and α-ketoglutarate (α-KG). The SSP consists of three successive enzymatic reactions. Phosphoglycerate dehydrogenase (PHGDH) catalyzes the first step and produces 3-phosphohydroxypyruvate (3-PPyr) by NAD+-coupled oxidation of 3-PG. Next, 3-PPyr is converted in phosphoserine by the phosphoserine aminotransferase 1 (PSAT-1) and then into serine by the action of phosphoserine phosphatase (PSPH). Finally, Ser can be converted into glycine by SHMT.

Elevated rates of SSP have been observed in neoplastic tissues of different origins (Snell & Weber, Biochem J. (1986) 233:617-620; DeBerardinis, Cell Metab. (2011) 14:285-286) and have been linked with tumorigenesis (DeBerardinis, Cell Metab. (2011) 14:285-286) with PHGDH being the key enzyme. PHGDH was shown to be amplified/overexpressed in melanoma and breast cancer (Beroukhim et al., Nature. (2010) 463:899-8905; Locasale et al., Nat Genet. (2011) 43:869-874; Possemato et al., Nature. (2011) 476:346-350). In addition, recent studies identified several factors as activators of the SSP in cancer cells which also determine cancer pathogenesis, such as the general control nonderepressible 2 kinase (GCN2) leading to expression of the activating transcription factor 4 (ATF4). Similarly, ATF4 can also be induced by the transcription factor nuclear factor erythroid-2-related factor 2 (NRF2) in human non-small-cell lung cancer (Wang et al., Neoplasia. (2013) 15:989-997; DeNicola et al., Nat Genet. (2015) 47:1475-1481). Also MYC activates the SSP by transcriptional upregulation of the expression of SSP enzymes under deprivation of glucose or glutamine (Sun et al., Cell Res. (2015) 25:429-444). Most importantly, a recent study demonstrated that hypoxia induces the expression of SSP enzymes, and this phenomenon is mediated by HIF-1 and HIF-2 in a large panel of breast cancer cell lines (Samanta et al., Cancer Res. (2016) 76:4430-4442). Finally, it was reported that tumor suppressors PKC-ζ and p53 repress the expression of PHGDH (Ma et al., Cell. (2013) 152:599-611; Ou et al., J Biol Chem. (2015) 290:457-466; Maddocks et al., Nature. (2013) 493:542-546). Thus, deficiency of PKC-ζ or p53 in cancer cells promotes the activity of PHGDH and drives the SSP.

The knockdown of PHGDH inhibited the growth of cancer cell lines that harbor PHGDH amplification and/or PHGDH overexpression but had no effect on lines expressing PHGDH at a normal level (Luo, Breast Cancer Res. (2011) 13:317; Possemato et al., Nature. (2011) 476:346-350). A negative-selection RNAi screening using a human breast cancer xenograft model at an orthotopic site in mouse was developed by Possemato et al. in 2011 for identifying novel cancer targets (Possemato et al., Nature. (2011) 476:346-350). This method highlighted PHGDH as a gene required for in vivo tumorigenesis and breast cancer progression (Samanta et al., Cancer Res. (2016) 76:4430-4442) and that this gene is localized in a genomic region of recurrent copy number gain in breast cancer. Subsequently, it was shown that the most abundantly expressed SSP enzymes in basal-like TNBC tissues was PHGDH and that the expression levels of PHGDH were inversely correlated with clinical prognostic factors (Noh et al., Tumour Biol. (2014) 35:4457-4468; Ravez et al., J Med Chem. (2017) 60 (4): 1227-1237). Also Knockdown of PHGDH in melanoma cells selectively inhibited the growth of cells that exhibit PHGDH amplification versus those that lack this amplification (Locasale et al., Nat Genet. (2011) 43:869-874; Mullarky et al., Pigment Cell Melanoma Res. (2011) 24:1112-1115). The prognostic significance of amplification/overexpression of PHGDH has clearly been demonstrated for colon cancer (Yoon et al., Oncology. (2015) 89:351-359; Jia et al., Transl Oncol. (2016) 9:191-196), glioma (Liu et al., J Neurooncol. (2013) 111:245-255), cervical adenocarcinoma (Jing et al., Cancer Biol Ther. (2015) 16:541-548) and lung adeno carcinoma (DeNicola et al., Nat Genet. (2015) 47:1475-1481; Amelio et al., Oncogene. (2014) 33:5039-5046). In thyroid cancer it was shown that a B-Raf V600E mutation was associated with a higher rate of PHGDH expression compared to non-mutant cases (Chen et al., Int J Mol Med. (2015) 36:1607-1614; Sun et al., J Transl Med. (2016) 14:168). Interestingly, in leukemia an increase in oxidative stress upon inhibition of glutamine metabolism was identified as the trigger of the up-regulation of PHGDH. Silencing of PHGDH inhibited leukemia cell growth, thereby identifying serine as a key pro-survival factor (Polet et al., Oncotarget. (2016) 7:1765-1776). Most recently, it was demonstrated that PHGDH catalyzes NADH-dependent reduction of α-ketoglutarate to the oncometabolite D-2-hydroxyglutarate (D-2HG) (Fan et al., ACS Chem Biol. (2015) 10:510-516). Originally D-2HG was identified as an oncometabolite leading to inhibition of several de-methylases thereby changing the epigentic landscape in tumor cells (Prensner & Chinnaiyan, Nature Medicine (2011) 17:291-293). D-2HG is produced in large amounts by isocitrate dehydrogenase mutants in glioma (Xu et al., Cancer Cell. (2011) 19:17-30; Rossetto et al., Rev Neurol (Paris) (2011) 167:699-703) and acute myeloid leukemia (Ward et al., Cancer Cell. (2010) 17:225-234; Ward et al., Oncogene. (2012) 31:2491-2498). Most interestingly, in breast cancer PHGDH was identified as an enzymatic driver of D-2HG production (Fan et al., ACS Chem Biol. (2015) 10:510-516). Terunuma and colleagues performed a detailed metabolic profiling of human breast tumors and uncovered intrinsic metabolite signatures in these tumors using an untargeted discovery approach and validation of key metabolites. D-2HG accumulated at high levels in those breast cancer tumors where MYC pathway activation was observed. Most importantly, MYC-driven accumulation of D-2HG is associated with a poor prognosis in breast cancer (Terunuma et al., J Clin Invest. (2014) 124:398-412). As it was shown that MYC-among others-regulate enzymes of the glycolytic pathway (Stine et al., Cancer Discov. (2015) 5:1024-39) PHGDH amplification and/or overexpression in breast cancer could potentially influence cell physiology by overproduction of D-2HG in a similar way (e.g. DNA methylation) as it was shown for glioma and AML (see above).

The mechanism(s) by which PHGDH supports tumorigenesis might be manifold but the enzymatic function of PHGDH is a prerequisite to essentially contribute to cell proliferation, invasion, and tumorigenicity of cancer cells. All these data strongly support PHGDH as an attractive drug target in tumors that overexpress PHGDH or exhibit PHGDH gene amplification.

Indole-2-carboxamide based NAD⁺-competitive PHGDH inhibitors have been disclosed in 2015 and published in 2016 illustrating fragment based drug discovery at AstraZenca (Fuller at al., Drug discovery today (2016), 21 (8), 1272-83). These compounds lack cellular potency.

WO 2018/167019 discloses tosylacetate based compounds that are potent and selective PHGDH inhibitors with nanomolar biomarker modulation.

Weinstabl et al., J. Med. Chem., 2019, 62, 7976-7997 reports a tosylacetate based carboxylic acid that is a selective and potent PHGDH inhibitor as well as its ester prodrug. In WO 2017/156179, RAZE THERAPEUTICS INC. describes indole based PHGDH inhibitors.

However, no orally bioavailable PHGDH inhibitor has been reported. Moreover, no prior art compound is known to inhibit PHGDH and to exhibit a good permeability profile at the same time.

Therefore, the aim of the present invention is to provide new compounds that can inhibit PHGDH, while also being permeable.

### DETAILED DESCRIPTION OF THE INVENTION

It has now been surprisingly found that compounds of formula (I) as defined hereinbelow act as inhibitors of PHGDH and also possess good oral bioavailability and permeability profiles. Thus, the compounds according to the invention may be used for example for the treatment of diseases characterised by excessive or abnormal cell proliferation, such as cancer.

The present invention therefore relates to a compound of formula (I):

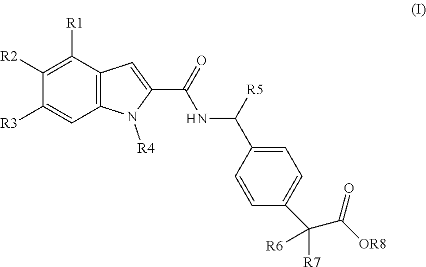

wherein:
R1, R2 and R3 are each independently selected from the group consisting of: hydrogen, C₁₋₃alkyl and halogen;
R4 is C₁₋₄alkyl;
R5 is C₁₋₃alkyl or hydroxy-C₁₋₃alkyl;
R6 and R7 together form a heterocyclyl ring containing one or more moieties selected from the group consisting of: —O—, —N(H)—, —N(COR^{a})—, —N(SO₂R^{a})—, —S—, —S(O)— and —S(O)₂—, wherein said heterocyclyl ring is optionally substituted by one or more substituents, each independently selected from —C(O)C₁₋₃alkyl and —N(H)COR^{a};
R8 is hydrogen or C₁₋₃alkyl;
R^{a} is selected from the group consisting of: C₁₋₃alkyl, —NH₂, —N(H)C₁₋₃alkyl and —N(C₁₋₃alkyl)₂;
or a pharmaceutically acceptable salt thereof.

The compounds of the invention have chiral centres. Although not separately depicted, e.g. in schemes and tables below, all stereoisomers of such compounds are meant to be embodiments of the invention and shall be deemed to be specifically disclosed, i.e. the compound as depicted, e.g. in schemes and tables, the corresponding enantiomer and/or diastereoisomers not specifically depicted in the tables and the racemate of both enantiomers are separate embodiments of the invention. The preferred embodiments are the compounds disclosed in the examples.

In an aspect, the present invention relates to compounds of formula (I′):

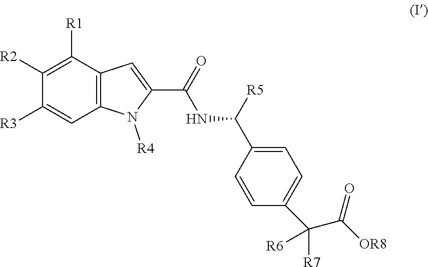

It is to be understood that formula (I′) is a subset of formula (I) and that the expression “compound(s) of formula (I)”, or grammatical variants thereof, also and equally refers to compound(s) of formula (I′) unless stated otherwise. Furthermore, any aspect or embodiment of the invention illustrated with reference to one or more compound(s) of formula (I) is also and equally applicable to compound(s) of formula (I′).

### Preferred Embodiments

In another aspect of the present invention, at least one of R1, R2 and/or R3 is halogen.

In another aspect, at least one of R1 and/or R2 is halogen.

In another aspect, R1 is halogen.

In another aspect, R2 is hydrogen or halogen.

In another aspect, R1 and R2 are each independently hydrogen or halogen.

In another aspect, R1 and R2 are each independently selected from the group consisting of: hydrogen, fluorine and chlorine.

In another aspect, R1 is halogen and R3 is C₁₋₃alkyl.

In another aspect:
R1 and R2 are both halogen;
R1 is halogen and R2 is hydrogen;
R1 is hydrogen and R2 is halogen; or
R1 and R2 are both hydrogen.

In another aspect:
R1 and R2 are fluorine;
R1 and R2 are chlorine;
R1 is fluorine and R2 is chlorine;
R1 is chlorine and R2 is fluorine;
R1 is chlorine and R2 is hydrogen;
R1 is hydrogen and R2 is chlorine;
R1 is fluorine and R2 is hydrogen; or
R1 is hydrogen and R2 is fluorine.

In another aspect, R3 is C₁₋₃alkyl.

In another aspect, R3 is methyl.

In another aspect, R4 is methyl.

In another aspect, R5 is methyl or —CH₂OH.

In another aspect, R5 is methyl.

In another aspect, R3, R4 and R5 are methyl.

In another aspect, the stereogenic centre at the carbon atom to which R5 is bound is in the (R) configuration.

In another aspect, R6 and R7 together form a heterocyclyl ring containing a moiety selected from the group consisting of: —O—, —N(H)—, —N(COR^{a})—, —N(SO₂R^{a})—, —S—, —S(O)— and —S(O)₂—, wherein said heterocyclyl ring is optionally substituted by one or more substituents, each independently selected from —C(O)C₁₋₃alkyl and —N(H)COR^{a}.

In another aspect, R6 and R7 together form a 3-7 membered saturated heterocyclyl ring containing one or more moieties selected from the group consisting of: —O—, —N(H)—, —N(COR^{a})—, —N(SO₂R^{a})—, —S—, —S(O)— and —S(O)₂—, wherein said heterocyclyl ring is optionally substituted by one or more substituents, each independently selected from —C(O)C₁₋₃alkyl and —N(H)COR^{a}.

In another aspect of the present invention, R6 and R7 together form a heterocyclyl ring containing a moiety selected from the group consisting of: —O—, —N(H)— and —S—, wherein said heterocyclyl ring is optionally substituted by one or more substituents, each independently selected from —C(O)C₁₋₃alkyl and —N(H)COR^{a}.

In another aspect, R6 and R7 together form a heterocyclyl ring containing one or more moieties selected from the group consisting of: —O—, —N(H)—, —N(COR^{a})—, —N(SO₂R^{a})—, —S—, —S(O)— and —S(O)₂—.

In another aspect, R6 and R7 together form a 3-7 membered saturated heterocyclyl ring containing a moiety selected from the group consisting of: —O—, —N(H)— and —S—, wherein said heterocyclyl ring is optionally substituted by one or more substituents, each independently selected from —C(O)C₁₋₃alkyl and —N(H)COR^{a}.

In another aspect, R6 and R7 together form a 3-7 membered saturated heterocyclyl ring containing a moiety selected from the group consisting of: —O—, —N(H)— and —S—.

In another aspect, R6 and R7 together form a 3-7 membered saturated heterocyclyl ring containing an oxygen atom, wherein said heterocyclyl ring is optionally substituted by one or more substituents, each independently selected from —C(O)C₁₋₃alkyl and —N(H)COR^{a}.

In another aspect, R6 and R7 together form a 3-7 membered saturated heterocyclyl ring containing an oxygen atom.

In another aspect, R6 and R7 together form a tetrahydropyran ring.

In another aspect, R6 and R7 together form:

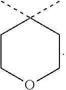

In the latter aspect and other aspects with the same notation, it is understood that dotted lines depict the attachment points of R6 and R7 to the rest of the compound of formula (I).

In another aspect, R8 is hydrogen.

In another aspect, R3, R4 and R5 are methyl, and R6 and R7 together form:

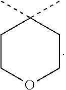

In another aspect, R3, R4 and R5 are methyl, R8 is hydrogen and R6 and R7 together form:

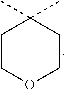

In another aspect, the compound of formula (I) or the pharmaceutically acceptable salt thereof is of formula (II):

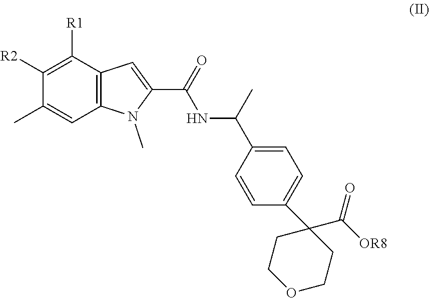

It is to be understood that formula (II) is a subset of formula (I) and that the expression “compound(s) of formula (I)”, or grammatical variants thereof, also and equally refers to compound(s) of formula (II) unless stated otherwise. Furthermore, any aspect or embodiment of the invention illustrated with reference to one or more compound(s) of formula (I) is also and equally applicable to compound(s) of formula (II).

In an aspect of formula (II), R8 is hydrogen.

In an embodiment, the compound of the present invention is selected from:

| Example | Structure |
|---|---|
| 1 | 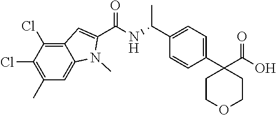 [SMILES: Cc1cc2c(cc(C(=O)N[C@H](C)c3ccc(C4(C(=O)O)CCOCC4)cc3)n2C)c(Cl)c1Cl] |
| 2 | 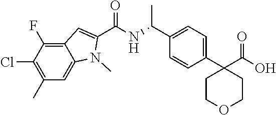 [SMILES: Cc1cc2c(cc(C(=O)N[C@H](C)c3ccc(C4(C(=O)O)CCOCC4)cc3)n2C)c(F)c1Cl] |
| 3 | 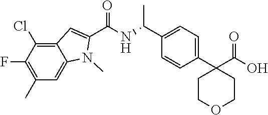 [SMILES: Cc1cc2c(cc(C(=O)N[C@H](C)c3ccc(C4(C(=O)O)CCOCC4)cc3)n2C)c(Cl)c1F] |
| 4 | 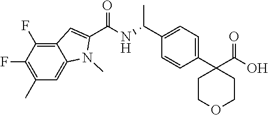 [SMILES: Cc1cc2c(cc(C(=O)N[C@H](C)c3ccc(C4(C(=O)O)CCOCC4)cc3)n2C)c(F)c1F] |
| 5 | 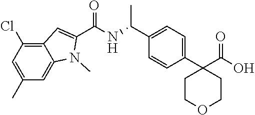 [SMILES: Cc1cc(Cl)c2cc(C(=O)N[C@H](C)c3ccc(C4(C(=O)O)CCOCC4)cc3)n(C)c2c1] |
| 6 | 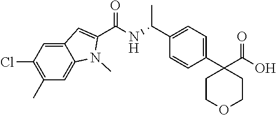 [SMILES: Cc1cc2c(cc1Cl)cc(C(=O)N[C@H](C)c1ccc(C3(C(=O)O)CCOCC3)cc1)n2C] |

or a pharmaceutically acceptable salt thereof.

It is understood that all tautomers of compound of formula (I), including all its aspects and embodiments, are herein disclosed and part of the invention, independently of the one that is depicted.

All synthetic intermediates generically defined as well as specifically disclosed herein and their salts are also part of the invention.

All individual synthetic reaction steps as well as reaction sequences comprising these individual synthetic reaction steps, both generically defined or specifically disclosed herein, are also part of the invention.

The present invention further relates to hydrates, solvates, polymorphs, co-crystals, metabolites, derivatives, isomers and prodrugs of a compound of formula (I), including all its aspects and embodiments.

The present invention further relates to a hydrate of a compound of formula (I), including all its aspects and embodiments.

The present invention further relates to a solvate of a compound of formula (I), including all its aspects and embodiments.

The present invention further relates to a pharmaceutically acceptable salt of a compound of formula (I), including all its aspects and embodiments.

The present invention further relates to a pharmaceutically acceptable salt of a compound of formula (I), including all its aspects and embodiments, with an organic or organic acids or bases.

### Medical Uses and Methods of Treatment

The present invention is directed to PHGDH inhibitors, particularly compounds of formula (I), including all its aspects and embodiments, which can be useful in the prevention and/or treatment of a disease and/or condition wherein the inhibition of PHGDH could be or is of therapeutic benefit, including but not limited to the treatment and/or prevention of cancer. In another aspect the invention relates to a compound of formula (I)—or a pharmaceutically acceptable salt thereof—for use as a medicament.

In another aspect the invention relates to a compound of formula (I)—or a pharmaceutically acceptable salt thereof—for use in a method of treatment of the human or animal body.

In another aspect the invention relates to a compound of formula (I)—or a pharmaceutically acceptable salt thereof—for use in the treatment and/or prevention of a disease and/or condition wherein the inhibition of PHGDH could be or is of therapeutic benefit.

In another aspect the invention relates to a compound of formula (I)—or a pharmaceutically acceptable salt thereof—for use in the treatment and/or prevention of cancer, infections, inflammations or autoimmune diseases.

In another aspect the invention relates to a compound of formula (I)—or a pharmaceutically acceptable salt thereof—for use in a method of treatment and/or prevention of cancer, infections, inflammations or autoimmune diseases in the human or animal body.

In another aspect the invention relates to the use of a compound of formula (I)—or a pharmaceutically acceptable salt thereof—for preparing a pharmaceutical composition for the treatment and/or prevention of cancer, infections, inflammations or autoimmune diseases.

In another aspect the invention relates to a compound of formula (I)—or a pharmaceutically acceptable salt thereof—for use in the treatment and/or prevention of cancer.

In another aspect the invention relates to a compound of formula (I)—or a pharmaceutically acceptable salt thereof—for use in a method of treatment and/or prevention of cancer in the human or animal body.

In another aspect the invention relates to the use of a compound of formula (I)—or a pharmaceutically acceptable salt thereof—for preparing a pharmaceutical composition for the treatment and/or prevention of cancer.

In another aspect the invention relates to a compound of formula (I)—or a pharmaceutically acceptable salt thereof—for use in the treatment and/or prevention of a hematological cancer.

In another aspect the invention relates to a compound of formula (I)—or a pharmaceutically acceptable salt thereof—for use in the treatment and/or prevention of glioma, breast cancer, melanoma, non-small cell lung cancer (NSCLC), colorectal cancer, cervical carcinoma, thyroid cancer, preferably BRAF mutated and leukemia.

In another aspect the invention relates to a compound of formula (I)—or a pharmaceutically acceptable salt thereof—for use in the treatment and/or prevention of p53 mutated cancer, MYC-driven cancers and/or cancers with a high level of D-2-hydroxyglutarate (D-2HG).

In another aspect the invention relates to the use of a compound of formula (I)—or a pharmaceutically acceptable salt thereof—for preparing a pharmaceutical composition for the treatment and/or prevention of a hematological cancer.

In another aspect the invention relates to the use of a compound of formula (I)—or a pharmaceutically acceptable salt thereof—for preparing a pharmaceutical composition for the treatment and/or prevention of glioma, breast cancer, melanoma, non-small cell lung cancer (NSCLC), colorectal cancer, cervical carcinoma, thyroid cancer, preferably BRAF mutated and leukemia.

In another aspect the invention relates to the use of a compound of formula (I)—or a pharmaceutically acceptable salt thereof—for preparing a pharmaceutical composition for the treatment and/or prevention of p53 mutated cancer, MYC-driven cancers and/or cancers with a high level of 2DHG.

In another aspect the invention relates to a method for the treatment and/or prevention of a disease and/or condition wherein the inhibition of PHGDH could be or is of therapeutic benefit comprising administering a therapeutically effective amount of a compound of formula (I)—or a pharmaceutically acceptable salt thereof—to a human being.

The expression “a disease and/or condition wherein the inhibition of PHGDH could be or is of therapeutic benefit” as used herein includes but is not limited to any condition where PHGDH is overexpressed, amplified, mutated or generally de-regulated.

In another aspect the invention relates to a method for the treatment and/or prevention of a disease or condition selected from the group consisting of: cancer, infections, inflammations and/or autoimmune diseases, wherein said method comprises administering a therapeutically effective amount of a compound of formula (I)—or a pharmaceutically acceptable salt thereof—to a human being.

In another aspect the invention relates to a method for the treatment and/or prevention of cancer comprising administering a therapeutically effective amount of a compound of formula (I)—or a pharmaceutically acceptable salt thereof—to a human being.

For example, the following cancers, tumors and other proliferative diseases may be treated with compounds of the invention, without being restricted thereto:
Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas,
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP).

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

The compounds of the invention may be used in therapeutic regimens in the context of first line, second line, or any further line treatments.

The compounds of the invention may be used for the prevention, short-term or long-term treatment of the above-mentioned diseases, optionally also in combination with radiotherapy and/or surgery.

### Pharmaceutical Compositions

The present invention also provides a pharmaceutical composition comprising the compound of formula (I) or the pharmaceutically acceptable salt as defined above, a pharmaceutically acceptable excipient and optionally one or more further pharmacologically active substance(s).

In another aspect the invention relates to a pharmaceutical composition comprising a compound of formula (I)—or a pharmaceutically acceptable salt thereof- and at least one pharmaceutically acceptable carrier.

In another aspect the invention relates to a pharmaceutical preparation comprising a compound of formula (I)—or a pharmaceutically acceptable salt thereof- and at least one other cytostatic and/or cytotoxic active substance.

Suitable pharmaceutical compositions for administering the compounds of the invention will be apparent to those with ordinary skill in the art and include for example tablets, pills, capsules, suppositories, lozenges, troches, solutions—particularly solutions for injection (s.c., i.v., i.m.) and infusion (injectables)—elixirs, syrups, sachets, emulsions, inhalatives or dispersible powders. The content of the compound(s) of the invention should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the compound(s) of the invention with known excipients, for example inert diluents, carriers, disintegrants, adjuvants, surfactants, binders and/or lubricants. The tablets may also comprise several layers. Coated tablets may be prepared accordingly by coating cores produced analogously to the tablets with substances and excipients normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the compound(s) of the invention may additionally contain a sweetener such as saccharine, cyclamate, glycerol or sugar and a flavour enhancer, e.g. a flavouring such as vanillin or orange extract. They may also contain excipients like suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of excipients like isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more compound(s) of the invention may for example be prepared by mixing the compound(s) with inert carriers such as lactose or sorbitol and packing them into gelatine capsules.

Suitable suppositories may be made for example by mixing with carriers provided for this purpose such as neutral fats or polyethyleneglycol or derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The pharmaceutical compositions are administered by the usual methods, preferably by oral or transdermal route, most preferably by oral route. For oral administration the tablets may of course contain, apart from the above-mentioned carriers, additional additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage range of the compounds of formula (I) applicable per day is usually from 1 mg to 2000 mg, preferably from 1 to 1000 mg, preferably from 1 to 100 mg.

The dosage for intravenous use is from 1 mg to 1000 mg with different infusion rates, preferably between 5 mg and 500 mg with different infusion rates.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts it may be advisable to divide them up into a number of smaller doses spread over the day.

The one or more further pharmacologically active substance(s) that is optionally present in the pharmaceutical composition as herein described may be chosen for example among the combination partners defined in the following paragraph.

### Combination Therapy

The compounds of the invention may be used on their own or in combination with one or several other pharmacologically active substances such as state-of-the-art or standard-of-care compounds, such as e.g. cell proliferation inhibitors, anti-angiogenic substances, steroids or immune modulators/checkpoint inhibitors, and the like.

In an aspect the invention relates to a compound of formula (I)—or a pharmaceutically acceptable salt thereof—for use as hereinbefore defined wherein said compound is administered before, after or together with at least one other cytostatic or cytotoxic active substance.

In another aspect the invention relates to a compound of formula (I)—or a pharmaceutically acceptable salt thereof—for use as hereinbefore defined wherein said compound is administered in combination with at least one other pharmacologically active substance, such as a cytostatic or cytotoxic active substance.

In another aspect the invention relates to a cytostatic or cytotoxic active substance prepared for being administered before, after or together with a compound of formula (I)—or a pharmaceutically acceptable salt thereof—for use as hereinbefore defined.

In another aspect the invention relates to a method for the treatment and/or prevention as hereinbefore defined comprising administering to a patient in need thereof a therapeutically effective amount of a compound of formula (I)—or a pharmaceutically acceptable salt thereof—before, after or together with at least one other cytostatic or cytotoxic active substance.

Pharmaceutically active substances, such as cytostatic and/or cytotoxic active substances, which may be administered in combination with the compounds according to the invention, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-) growth factor antibodies, (anti-) growth factor receptor antibodies and tyrosine kinase inhibitors, such as for example cetuximab, gefitinib, afatinib, nintedanib, imatinib, lapatinib, bosutinib, bevacizumab and trastuzumab); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine);

antitumour antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, FLT3 inhibitors, BRD4 inhibitors, IGF-1R inhibitors, TRAILR2 agonists, Bcl-XL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), t-cell engagers (e.g. bi-specific T-cell engagers (BiTEs®) like e.g. CD3×BCMA, CD3×CD33, CD3×CD19), PSMA×CD3), tumor vaccines and various chemotherapeutic agents such as amifostin, anagrelid, clodronat, filgrastin, interferon, interferon alpha, leucovorin, procarbazine, levamisole, mesna, mitotane, pamidronate and porfimer.

Most preferred are combinations with IAP activators, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3×BCMA, CD3×CD33, CD3×CD19, PSMA×CD3) and tumor vaccines.

When two or more substances or principles are to be used as part of a combined treatment regimen, they can be administered via the same route of administration or via different routes of administration, at essentially the same time (i.e. simultaneously, concurrently) or at different times (e.g. sequentially, successively, alternately, consecutively, or according to any other sort of alternating regime).

When the substances or principles are to be administered simultaneously via the same route of administration, they may be administered as different pharmaceutical formulations or compositions or as part of a combined pharmaceutical formulation or composition. Also, when two or more active substances or principles are to be used as part of a combined treatment regimen, each of the substances or principles may be administered in the same amount and according to the same regimen as used when the compound or principle is used on its own, and such combined use may or may not lead to a synergistic effect.

However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmacological or therapeutic effect.

Of course, the above includes the preparation and methods of preparing, the compounds of the invention for the combined use with the above combination partners. Also included are the preparation, and methods of preparing, the above-mentioned combination partners for the combined use with the compounds of the invention.

Furthermore, the invention also encompasses kits comprising at least one compound of the invention and one or more other components selected from the group consisting of other drugs used for the treatment of the diseases and disorders as described above, and devices as described below.

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

All different depictions of Rx with superscript or subscript, such as R^{x} or Rₓ shall refer to and be understood as Rx, e.g. R1 or R¹ should refer to R1.

In the groups, radicals, or moieties defined below, the number of carbon atoms is often specified preceding the group, for example, C₁₋₆-alkyl means an alkyl group or radical having 1 to 6 carbon atoms. In particular, the use of the prefix C_{x-y}, wherein x and y each represent a natural number (x<y), indicates that the chain or ring structure or combination of chain and ring structure as a whole, specified and mentioned in direct association, may consist of a maximum of y and a minimum of x carbon atoms.

The indication of the number of members in groups that contain one or more heteroatom(s) (e.g. heterocyclyl) relates to the total number of atoms of all the ring members or chain members or the total of all the ring and chain members.

In general, for combined groups comprising two or more subgroups (e.g. hydroxyalkyl), the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In general, in groups like OH, NH₂, S(O), S(O)₂, CN (cyano), COOH, CF₃ or the like, the skilled artisan can see the radical attachment point(s) to the molecule from the free valences of the group itself.

In case a compound of the present invention is depicted in the form of a chemical name and as a formula, in case of any discrepancy the formula shall prevail.

As it will be clear to the person skilled in the art, the radical attachment point(s) to the molecule from the free valences of the group itself can be indicated for example with a dash (“—”), an asterisk (“*”) or a dotted line (“ . . . ”). An asterisk may be used in sub-formulas to indicate the bond which is connected to the core molecule as defined.

The term “halogen” denotes fluorine, chlorine, bromine and iodine. Preferably, “halogen” refers to fluorine or chlorine.

The term “C₁₋ₙ-alkyl”, wherein n is an integer selected from 2, 3, 4, 5 or 6, preferably 3, 4, or 5, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Preferably, “C₁₋₄alkyl” as used herein refers to: methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃) CH₂CH₃), 2-methyl-2-propyl (tert-butyl; t-Bu; —C(CH₃)₃).

Preferably, “C₁₋₄alkyl” as used herein refers to: methyl (Me; —CH₃).

Preferably, “C₁₋₃alkyl” as used herein refers to: methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂).

Preferably, “C₁₋₃alkyl” as used herein refers to: methyl (Me; —CH₃).

By the terms propyl, butyl, etc. without any further definition are meant saturated hydrocarbon groups with the corresponding number of carbon atoms, wherein all isomeric forms are included.

The above definition for alkyl also applies if alkyl is a part of another (combined) group such as for example hydroxy-C_{x-y}alkyl.

The term “hydroxy-C₁₋₃alkyl” refers to a C₁₋₃alkyl as defined above, wherein any one or more hydrogen atoms of the hydrocarbon chain is replaced by —OH.

The term “heterocyclyl” or “heterocyclyl ring” means a saturated or unsaturated mono- or polycyclic ring system optionally comprising aromatic rings, containing one or more heteroatoms selected e.g. from N, O, S, SO or SO₂ consisting of 3 to 14 ring atoms wherein none of the heteroatoms is part of the aromatic ring. The term “heterocyclyl” is intended to include all the possible isomeric forms.

Thus, the term “heterocyclyl” or “heterocyclyl ring” includes the following exemplary structures (not depicted as radicals as each form is optionally attached through a covalent bond to any atom so long as appropriate valences are maintained):

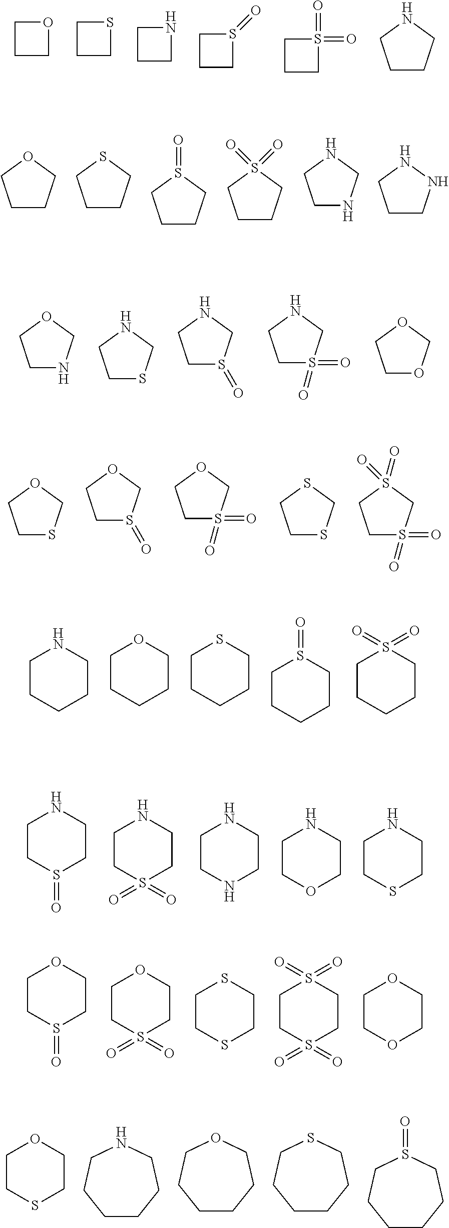

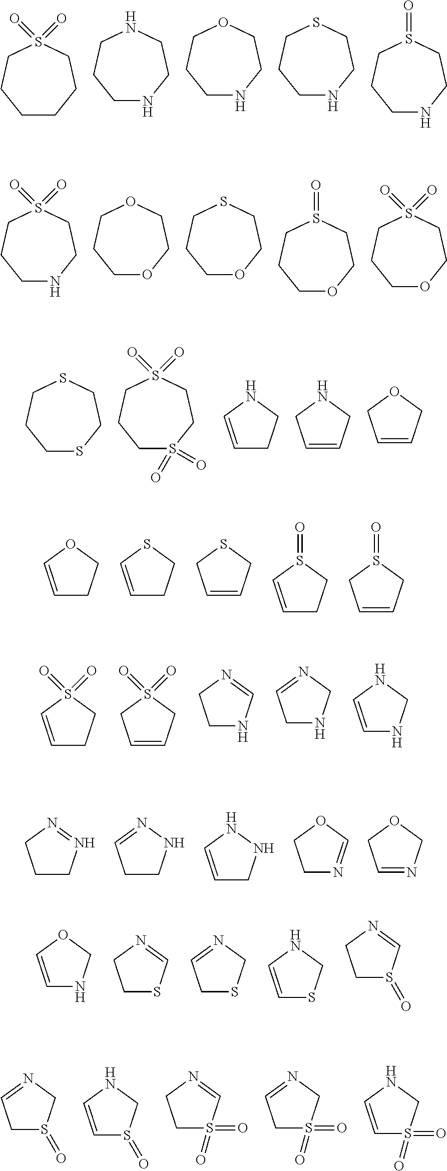

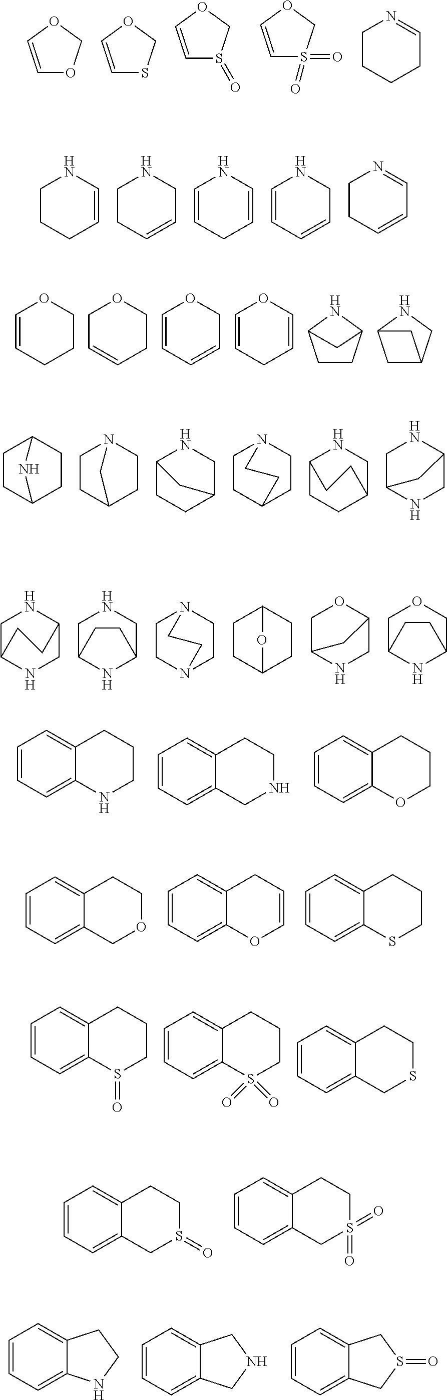

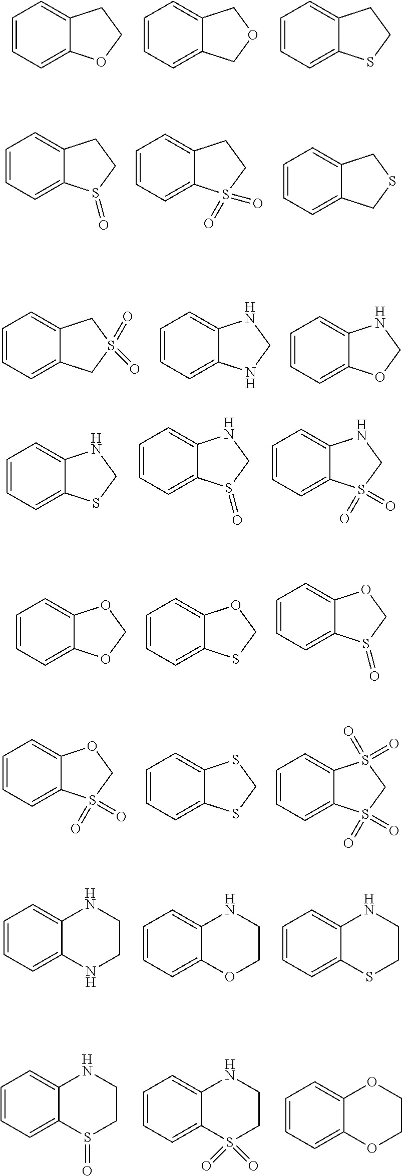

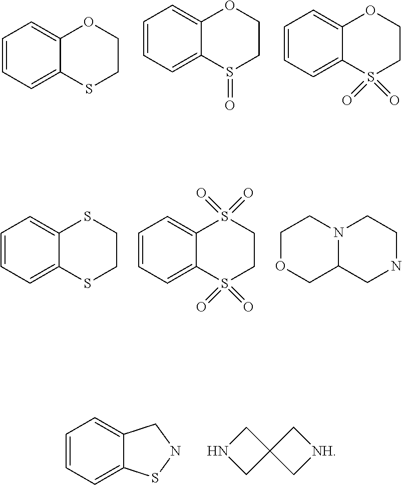

By unsaturated, it is meant that there is at least one double bond in the heterocyclyl ring system in question, but no heteroaromatic system is formed. In bicyclic heterocyclyl rings two rings are linked together so that they have at least two (hetero) atoms in common. In spiro-heterorings one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system.

Preferably, heterocyclyls are 3 to 7 membered, monocyclic, saturated and have one heteroatom selected from oxygen, nitrogen and sulfur.

Preferred heterocyclyls are: piperazinyl, piperidinyl, morpholinyl, pyrrolidinyl, azetidinyl, tetrahydropyranyl, tetrahydrofuranyl.

The term “substituted” as used herein, means that one or more hydrogens on the designated atom are replaced by one or more groups selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases, or by resolution of a racemic mixture using an appropriate resolving agent, e.g. by means of diastereomeric salt formation of the racemic compound with optically active acids or bases, subsequent resolution of the salts and release of the desired compound from the salt, or by derivatization of the corresponding racemic compounds with optically active chiral auxiliary reagents, subsequent diastereomer separation and removal of the chiral auxiliary group, or by kinetic resolution of a racemate (e.g. by enzymatic resolution); by enantioselective crystallization from a conglomerate of enantiomorphous crystals under suitable conditions, or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

Many of the terms given above may be used repeatedly in the definition of a formula or group and in each case have one of the meanings given above, independently of one another.

The term “a therapeutically effective amount” for the purposes of this invention refers to a quantity of substance that is capable of obviating symptoms of illness or of preventing or alleviating these symptoms, or which prolong the survival of a treated patient.

List of abbreviations

| | |
|---|---|
| Bu | butyl |
| TLC | thin layer chromatography |
| DCM | dichloromethane |
| DMEM | Dulbecco's Modified Eagle Medium |
| DMF | N,N-dimethylformamide |
| DMSO | dimethylsulphoxide |
| EDTA | ethylenediaminetetraacetic acid |
| ESI | electron spray ionization |
| Et | ethyl |
| EtOAc | ethyl acetate |
| EtOH | ethanol |
| h | hour |
| HATU | O-(7-azabenzotriazol-1-yl)-N,N,N′,N′-tetramethyl- |
| | uronium hexafluorophosphate |
| HEPES | (4-(2-hydroxyethyl)-1-piperazineethanesulfonic acid) |
| HOAT | 1-hydroxy-7-azabenzotriazole |
| HPLC | high performance liquid chromatography |
| HTP | HEPES transport buffer |
| i | iso |
| LC | liquid chromatography |
| Me | methyl |
| MeCN | acetonitrile |
| MeOH | methanol |
| min | minutes |
| MS | mass spectrometry |
| NAD⁺ | nicotinamide adenine dinucleotide (oxidized form) |
| NADH | nicotinamide adenine dinucleotide (reduced form) |
| n.a. | not available |
| Ph | phenyl |
| Pr | propyl |
| Pyr | pyruvate |
| RP | reversed phase |
| rt | ambient temperature |
| tBu | tert.-butyl |
| temp. | temperature |
| Tert. | tertiary |
| THF | tetrahydrofuran |
| t_{Ret.} | retention time (HPLC) |
| TRIS | tris(hydroxymethyl)-aminomethane |
| UV | ultraviolet |

### General Considerations on the Synthetic Schemes

The compounds according to the present invention and their intermediates may be obtained using methods of synthesis which are known to the one skilled in the art and described in the literature of organic synthesis. Preferably, the compounds according to the invention are prepared by the methods of synthesis described hereinafter in which the substituents of the general formulae have the meanings given hereinbefore. These methods are intended as an illustration of the invention without restricting its subject matter and the scope of the compounds claimed to these examples. Where the preparation of starting compounds is not described, they are commercially available or their synthesis is described in the prior art or they may be prepared analogously to known prior art compounds or methods described herein. Substances described in the literature are prepared according to or in analogy to the published methods of synthesis. It is to be understood that compounds of a certain formula may be converted into different compounds of the same formula. In some cases, the order in carrying out the reaction steps may be varied. Variants of the reaction methods that are known to the one skilled in the art but not described in detail here may also be used. Any functional groups in the starting materials or intermediates may be protected using conventional protecting groups. These protecting groups may be cleaved again at a suitable stage within the reaction sequence using methods familiar to the one skilled in the art.

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

The compounds according to the invention are named in accordance with CAS rules using the software Autonom (Beilstein).

If a chemical structure is depicted without exact configuration of a stereo center, e.g. of an asymmetrically substituted carbon atom, then both configurations shall be deemed to be included and disclosed in such a representation. The representation of a stereo center in racemic form shall always deem to include and disclose both enantiomers (if no other defined stereo center exists) or all other potential diastereomers and enantiomers (if additional, defined or undefined, stereo centers exist).

Microwave reactions are carried out in an initiator/reactor made by Biotage or in an Explorer made by CEM or in Synthos 3000 or Monowave 3000 made by Anton Paar in sealed containers (preferably 2, 5 or 20 mL), preferably with stirring.

### Chromatography

The thin layer chromatography is carried out on ready-made silica gel 60 TLC plates on glass (with fluorescence indicator F-254) made by Merck.

The preparative high pressure chromatography (RP HPLC) of the example compounds according to the invention is carried out with columns made by Waters (names: XTerra Prep. MS C18, 5 μm, 30×100 mm or XTerra Prep. MS C18, 5 μm, 50×100 mm OBD or Symmetrie C18, 5 μm, 19×100 mm or Sunfire C18 OBD, 19×100 mm, 5 μm or Sunfire Prep C 10 μm OBD 50×150 mm or X-Bridge Prep C18 5 μm OBD 19×50 mm) or X-Bridge Prep C18 10 μm OBD 50×150 mm), Agilent (name: Zorbax SB-C8 5 μm PrepHT 21.2×50 mm) and Phenomenex (names: Gemini C18 5 μm AXIA 21.2×50 mm or Gemini C18 10 μm 50×150 mm). Different gradients of H₂O/acetonitrile or H₂O/MeOH are used to elute the compounds, while 0.1% HCOOH is added to the water (acidic conditions). For the chromatography under basic conditions H₂O/acetonitrile gradients are used as well, while the water is made alkaline as follows: 5 mL NH₄HCO₃ solution (158 g in 1 L H₂O) and 2 mL NH₃ (7 M in MeOH) are replenished to 1 L with H₂O.

The analytical HPLC (reaction control) of intermediate compounds is carried out using columns made by Agilent (names: Zorbax SB-C8, 5 μm, 21.2×50 mm or Zorbax SB-C8 3.5 μm 2.1×50 mm), Phenomenex (name: Gemini C18 3 μm 2×30 mm) and Waters (names: XBridge™ C18, 3.5 μm, 2.1×50 mm, XBridge™ C18, 5 μm, 2.1×50 mm, XBridge™ C18, 2.5 μm, 2.1×20 mm or Sunfire™ C18, 3.5 μm, 2.1×50 mm. The analytical equipment is also equipped with a mass detector in each case.

### HPLC-Mass Spectroscopy/UV-Spectrometry

The retention times/MS-ESI⁺ for characterizing the example compounds according to the invention are produced using an HPLC-MS apparatus (high performance liquid chromatography with mass detector). Compounds that elute at the injection peak are given the retention time t_{Ret.}=0.00.

### Method 1

| HPLC | Agilent 1100/1200 system | |
|---|---|---|
| MS | 1200 Series LC/MSD (API-ES +/− 3000 V, | |
| | Quadrupol, G6140) | |
| MSD signal | Scan pos 150-750, Scan neg 150-750 | |
| settings | | |
| column | YMC; Part. No. TA12S03-0302WT; Triart C18, 3 μm, | |
| | 12 nm; 30 × 2.0 mm column | |
| eluant | A: H2O + 0.11% formic acid | |
| | B: MeCN + 0.1% formic acid (HPLC grade) | |
| detection | UV 254 nm (bandwidth 10, reference off) | |
| signal | | |
| spectrum | range: 190-400 nm; step: 4 nm | |
| peak width | >0.005 min (0.1 s) | |
| injection | 0.5 μL standard injection | |
| flow | 1.4 mL/min | |
| column | 45° C. | |
| temperature | | |
| gradient | 0.0-1.0 min | 15% → 100% B |
| | 1.0-1.1 min | 100% B |
| | Stop time: 1.23 min | |

### Method 2

| HPLC | Agilent 1100/1200 system | |
|---|---|---|
| MS | 1200 Series LC/MSD (API-ES +/− 3000 V, | |
| | Quadrupol, G6140) | |
| MSD signal | Scan pos 150-750 | |
| settings | | |
| column | YMC; Part. No. TA12S03-0302WT; Triart C18, 3 μm, | |
| | 12 nm; 30 × 2.0 mm column | |
| eluant | A: H2O + 0.11% formic acid | |
| | B: MeCN + 0.1% formic acid (HPLC grade) | |
| detection | UV 254 nm (bandwidth 10, reference off) | |
| signal | | |
| spectrum | range: 190-400 nm; step: 4 nm | |
| peak width | >0.005 min (0.1 s) | |
| injection | 0.5 μL standard injection | |
| flow | 1.4 mL/min | |
| column | 45° C. | |
| temperature | | |
| gradient | 0.0-1.0 min | 15% → 100% B |
| | 1.0-1.1 min | 100% B |
| | Stop time: 1.23 min | |

### Method 3

| LC | Agilent Infinity 1290 series | |
|---|---|---|
| MS | Agilent 6150 Quadruple lcms(SQ) | |
| MSD signal settings | Scan pos/neg 100-1200 | |
| column | Aquity BEH C18 2.1 × 50 mm, 1.7 μm | |
| eluant | A: water + 0.1% formic acid | |
| | B: acetonitrile (HPLC grade) + 0.1% formic acid | |
| detection signal | UV 215/254 nm (bandwidth 4, reference off) | |
| spectrum | range: 200-400 nm; step: 2.0 nm | |
| peak width | >0.01 min (0.2 s) | |
| injection | 0.5 μL standard injection | |
| flow | 0.6 mL/min | |
| column temperature | 25° C. | |
| gradient | 0.0-0.4 min | 3% B |
| | 0.4-3.2 min | 3% → 98% B |
| | 3.2-3.8 min | 98% B |
| | 3.8-4.2 min | 98% → 3% B |
| | 4.2-4.5 min | 3% B |

### Method 4

| HPLC | Agilent 1100 system | |
|---|---|---|
| MS | 1200 Series LC/MSD (API-ES +/− 3000 V, | |
| | Quadrupol, G6130) | |
| MSD signal | Scan pos/neg 120-900 m/z | |
| settings | | |
| column | Waters, Xbridge C18, 2.5 μm, 2.1 × 20 mm column | |
| eluant | A: 20 mM NH₄HCO₃/NH₃ pH 9 | |
| | B: acetonitrile HPLC grade | |
| detection signal | 315 nm (bandwidth 170 nm, reference off) | |
| spectrum | range: 230-400 nm | |
| peak width | <0.01 min | |
| injection | 5 μL standard injection | |
| column | 60° C. | |
| temperature | | |
| flow | 1.00 mL/min | |
| gradient | 0.00-1.50 min | 10% → 95% B |
| | 1.50-2.00 min | 95% B |
| | 2.00-2.10 min | 95% → 10% B |

### Method 5

| UPLC-MS | Waters Acquity-UPLC-SQ Detector-2 | |
|---|---|---|
| MSD signal | Scan pos & Neg 100-1500 | |
| settings | Source Voltage: Capillary Vol (kV), 3.50, Cone (V): | |
| | 50 | |
| | Source Temp: Desolvation Temp (° C.): 350 | |
| | Source Gas Flow: Desolvation (L/Hr): 750, Cone (L/Hr): | |
| | 50 | |
| Column | AQUITY UPLC BEH C18 1.7 μm, 2.1 × 50 mm | |
| | Part. No.: 186002350 | |
| Eluent | A: 0.07% formic acid in acetonitrile | |
| | B: 0.07% formic acid in water | |
| Detection | Diode Array | |
| signal | | |
| Spectrum | Range: 200-400 nm; Resolution: 1.2 nm | |
| Sampling rate | 10 point/sec | |
| Injection | 0.5 μL standard injection | |
| Flow | 0.6 mL/min | |
| Column | 35° C. | |
| temperature | | |
| Gradient | 0.00-0.30 min | 97% B |
| | 0.30-2.20 min | 97% → 2% B |
| | 2.20-3.30 min | 2% B |
| | 3.30-4.50 min | 2% → 97% B |
| | 4.50-4.51 min | 9% B |

### Method 6

| HPLC | Agilent 1100/1200 system | |
|---|---|---|
| MS | 1200 Series LC/MSD (MM-ES + APCI +/− 4000 V, | |
| | Quadrupol, G6130) | |
| MSD signal | Scan pos 150-800, Scan neg 150-800 | |
| settings | | |
| column | Waters; Part. No. 186006028; XBridge BEH C18 XP, 2.5 | |
| | μm, 30 × 2.1 mm column | |
| eluant | A: 5 mM NH4HCO3/18 mM NH3 (pH = 9.2) | |
| | B: acetonitrile (HPLC grade) | |
| detection | UV 254 nm (bandwidth 8, reference off) | |
| signal | | |
| spectrum | range: 190-400 nm; step: 4 nm | |
| peak width | 0.0025 min (0.05 s) | |
| injection | 0.5 μL standard injection | |
| flow | 1.4 mL/min | |
| column | 45° C. | |
| temperature | | |
| gradient | 0.0-1.0 min | 15% → 95% B |
| | 1.0-1.3 min | 95% B |
| | Stop time: 1.3 min | |

### General Reaction Scheme Summarising the Synthesis Route

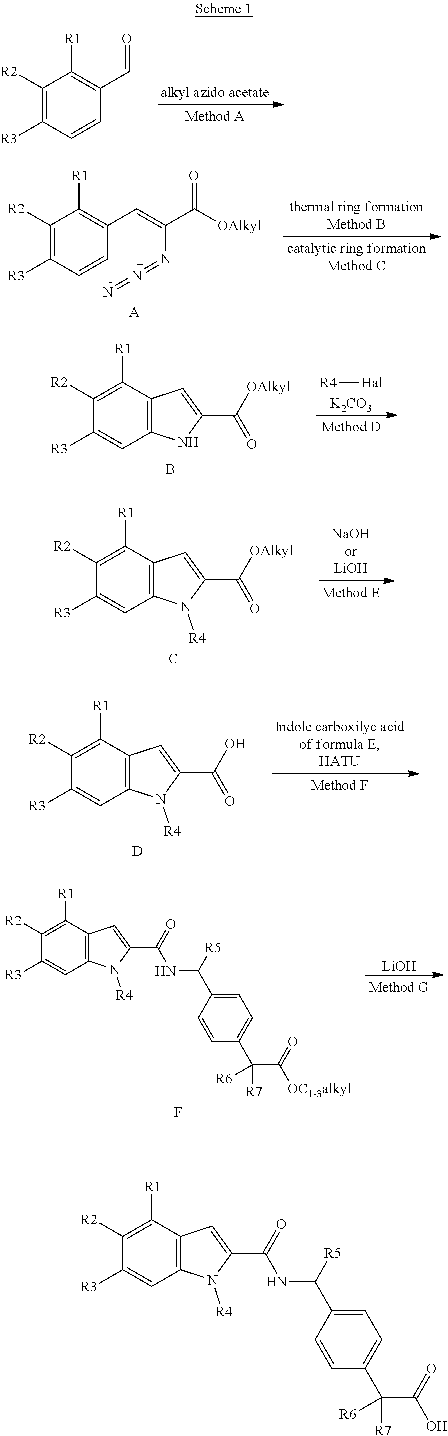

### Examples

Features and advantages of the present invention will become apparent from the following detailed examples which illustrate the fundamentals of the invention by way of example without restricting its scope.

### Synthesis of Indole Carboxylate Intermediates by Hemetsberger-Knittel Synthesis

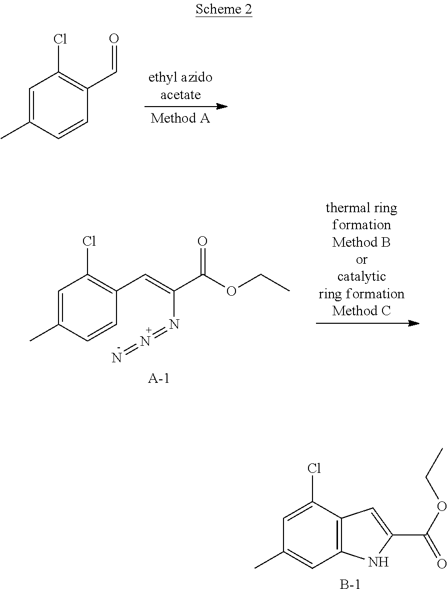

### Synthesis of A-1 (Method A)

To sodium ethoxide (25% in ethanol, 293.3 g, 906 mmol) and 2-chloro-4-methylbenzaldehyde (35.0 g, 226 mmol) ethyl azido acetate (116.8 g, 906 mmol) in THF (70 mL)/ethanol (700 mL) is added at −30° C. and stirred for 1 h at ambient temperature. Ice water is added, and the solid is collected by filtration.

The following azido esters are available in an analogous manner starting from different aldehydes.

| Intermediate | Structure | NMR | | |
|---|---|---|---|---|
| A-1 | 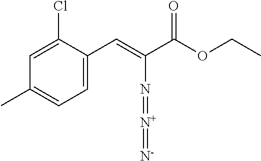 [SMILES: CCOC(=O)/C(=C/c1ccc(C)cc1Cl)N=[N+]=[N-]] | ¹H-NMR (400 MHz; CDCl₃): δ 8.07-8.09 (d, 1H J = 8 Hz); 7.29 (s, 1H); 7.24-7.260 (d, 1H, J = 8.8 Hz;); 7.09-7.11 (d, 1H, J = 8 Hz); 4.35-4.41(m, 2H); 2.34 (s, 3H); 1.38-1.42 (t, 3H). | | |
| A-2 | 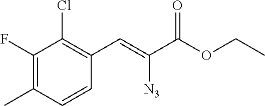 [SMILES: CCOC(=O)/C(=C/c1ccc(C)c(F)c1Cl)N=[N+]=[N-]] | ¹H-NMR (400 MHz; DMSO): δ 7.97-7.95 (d, 1H, J = 8 Hz); 7.37-7.33 (t, 1H, J = 14.8 Hz); 7.07 (s,1H); 3.88 (s, 3H); 2.30 (s, 3H). | | |
| A-3 | 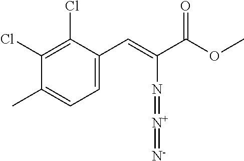 | 0.95 | not detectable due to chemical instability of the ionized species | Method 1 |
| A-4 | 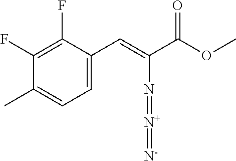 | 0.87 | 267 | Method 2 |

### Synthesis of B-1 (Method B)

A-1 (26.0 g, 265.7 mmol) in xylene (20 mL) is added to xylene (520 mL) at 160° C. over a period of 20 min, and stirred for 3 h at this temperature. The reaction mixture is concentrated in vacuo and triturated with pentane (100 mL).

### Synthesis of B-3 (Method C)

To A-3 (3.27 g, 11.4 mmol) in toluene (250 mL) rhodium (II) heptafluorobutyrate dimer (413 mg, 0.63 mmol) is added and stirred at 70° C. overnight. The reaction mixture is concentrated in vacuo, and the residue is purified by column chromatography. 10 The following indoles are available in an analogous manner applying the given synthetic method.

| | | syn- | | M | |
|---|---|---|---|---|---|
| Inter- | | thetic | tᵣₑₜ | + | |
| mediate | Structure | method | [min] | H | Method |
| B-1 | 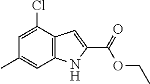 [SMILES: CCOC(=O)c1cc2c(Cl)cc(C)cc2[nH]1] | B | 2.79 | 238 | Method 3 |
| B-2 | 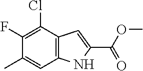 | B | 2.62 | 240 | Method 3 |
| B-3 | 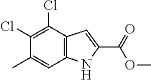 | C | 0.81 | 258 | Method 1 |
| B-4 | 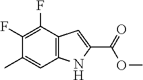 | C | 0.70 | 226 | Method 2 |

### Synthesis of Carboxylic Acid Intermediates

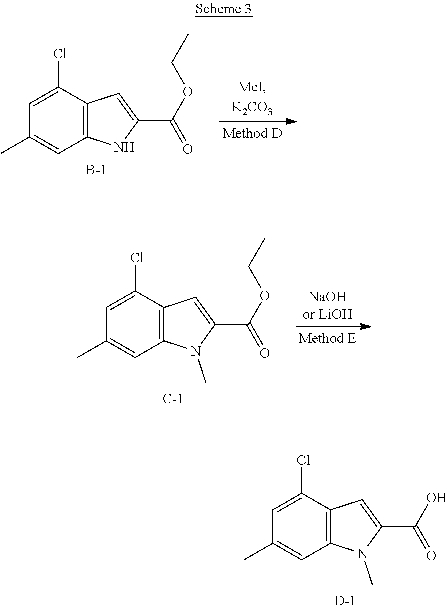

### Synthesis of C-1 (Method D)

To B-1 (12.0 g, 50.5 mmol) and K₂CO₃ (13.96 g, 101 mmol) in DMF (120 mL) methyl iodide (14.34 g, 101 mmol) is added at 0° C. and stirred for 4 h at ambient temperature. Ice water is added. The formed solid is collected by filtration and triturated subsequently with water and pentane.

The following indoles are prepared in an analogous manner starting from the corresponding indole carboxylate intermediate of formula B.

| Intermediate | Structure | tᵣₑₜ [min] | M + H | Method |
|---|---|---|---|---|
| C-1 | 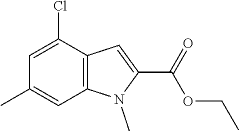 [SMILES: CCOC(=O)c1cc2c(Cl)cc(C)cc2n1C] | 3.12 | 252 | Method 3 |
| C-3 | 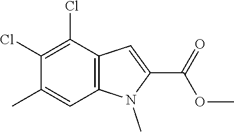 | 0.92 | 272 | Method 1 |
| C-4 | 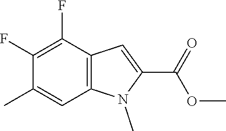 | 0.83 | 240 | Method 2 |

| Intermediate | Structure | NMR |
|---|---|---|
| C-2 | 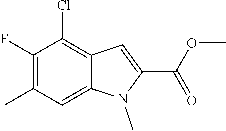 | ¹H-NMR (300 MHz; CDCl₃): δ 7.30 (s, 1H); 7.06- 7.08 (d, 1H, J = 5.1 Hz); 4.04 (s, 3H); 3.92(s, 3H); 2.45 (s, 3H) |

### Synthesis of D-1 (Method E)

To C-1 (12.0 g, 47.7 mmol) in THF (70 mL)/water (25 mL) lithium hydroxide monohydrate (8.01 g, 191 mmol) is added at 0° C. and stirred at ambient temperature for 2 h. 4 N HCl (10 mL) is added, and the mixture is extracted exhaustively with EtOAc. The combined organic layer is washed with water and brine, dried (MgSO₄), filtered and concentrated in vacuo. The residue is triturated with ether and pentane.

The following indole carboxylic acids are prepared in an analogous manner starting from the respective ester precursors of formula C.

| Intermediate | Structure | tᵣₑₜ [min] | M + H | M − H | Method |
|---|---|---|---|---|---|
| D-1 | 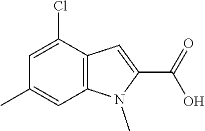 | 2.47 | | 222 | Method 3 |
| D-2 | 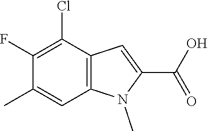 [SMILES: Cc1cc2c(cc(C(=O)O)n2C)c(Cl)c1F] | 2.47 | 240 | | Method 5 |
| D-3 | 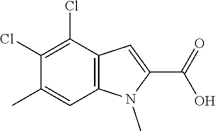 [SMILES: Cc1cc2c(cc(C(=O)O)n2C)c(Cl)c1Cl] | 0.74 | 258 | | Method 1 |
| D-4 | 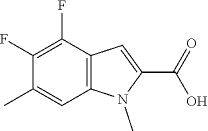 [SMILES: Cc1cc2c(cc(C(=O)O)n2C)c(F)c1F] | 0.64 | 226 | | Method 2 |

Compounds D-5 and D-6 can be prepared employing analogous methods to those described above for D-1, D-2, D-3 and D-4 starting from the corresponding halogen substituted benzylaldehydes.

| Intermediate | Structure |
|---|---|
| D-5 | 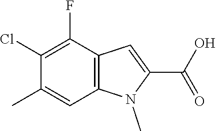 [SMILES: Cc1cc2c(cc(C(=O)O)n2C)c(F)c1Cl] |
| D-6 | 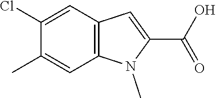 |

### Synthesis of the Benzylamine Building Block

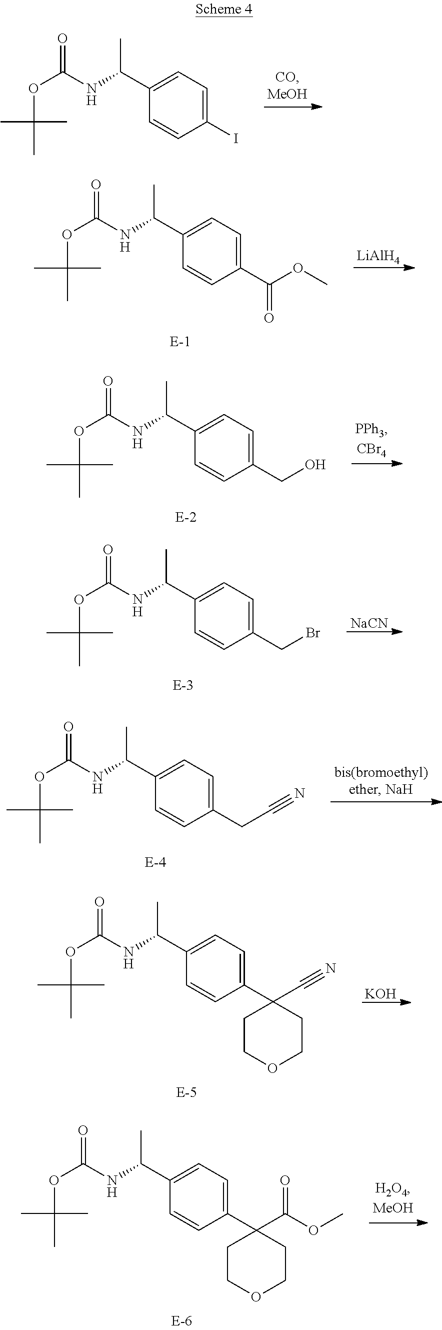

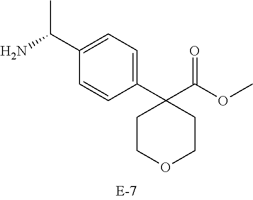 [SMILES: COC(=O)C1(c2ccc([C@@H](C)N)cc2)CCOCC1]

### Synthesis of E-1

To a stirred solution of [(R)-1-(4-iodophenyl)ethyl]carbamic acid tert.-butyl ester (100.0 g, 288.0 mmol, producible by the procedure given in WO2011076786) in MeOH (900 mL) triethylamine (101 mL, 720.0 mmol) is added under an argon atmosphere. 1,1′-Bis(diphenylphosphino) ferrocene]dichloropalladium (II), complex with dichloromethane (16.5 g, 20 mmol) is added, and the reaction mixture is stirred under a CO atmosphere (250 psi) for 16 h at 120° C. The mixture is filtered through a celite bed and concentrated in vacuo. The crude product is purified by column chromatography (SiO₂, 30% EtOAc in hexane).

| Intermediate | Structure | tᵣₑₜ [min] | M + H | Method |
|---|---|---|---|---|
| E-1 | 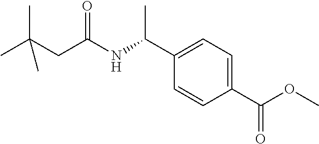 [SMILES: COC(=O)c1ccc([C@@H](C)NC(=O)CC(C)(C)C)cc1] | 2.23 | 224 (M − tBu) | Method 5 |

### Synthesis of E-2

To E-1 (63.0 g, 225.6 mmol) in dry THF (700 mL) lithium aluminium hydride (1 M in THF, 338.3 mL, 338.3 mmol) is added at 0° C. and stirred for 2 h at room temperature. Saturated aqueous Na₂SO₄ solution is added, and the mixture is filtered through a celite bed. The aqueous layer is extracted exhaustively with EtOAc. The combined organic layer is dried (Na₂SO₄), filtered, and concentrated in vacuo. The crude product is purified by column chromatography (SiO₂, 0-25% EtOAc in hexane).

| Intermediate | Structure | tᵣₑₜ [min] | M + H | Method |
|---|---|---|---|---|
| E-2 | 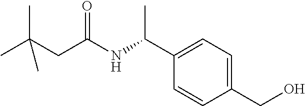 [SMILES: C[C@@H](NC(=O)CC(C)(C)C)c1ccc(CO)cc1] | 1.93 | 196 (M − tBu) | Method 5 |

### Synthesis of E-3

To E-2 (50.0 g, 199.0 mmol) in dry CH₂Cl₂ (500 mL), triphenylphosphine (78.3 g, 298.5 mmol) and CBr₄ (99.0 g, 298.5 mmol) are added at 0° C. and stirred for 5 h at room temperature. Water is added, and the aqueous layer is extracted exhaustively with CH₂Cl₂. The combined organic layer is dried (Na₂SO₄), filtered, and concentrated in vacuo. The crude product is purified by column chromatography (SiO₂, 10% EtOAc in hexane).

| Intermediate | Structure | tᵣₑₜ [min] | M + H | Method |
|---|---|---|---|---|
| E-3 | 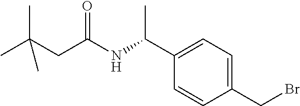 [SMILES: C[C@@H](NC(=O)CC(C)(C)C)c1ccc(CBr)cc1] | 2.37 | 258 (M − tBu) | Method 5 |

### Synthesis of E-4

To E-3 (35.0 g; 111.4 mmol) in DMF (350 mL), NaCN (10.92 g, 222.8 mmol) is added and stirred for 16 h at room temperature. The mixture is treated with water, the precipitate is collected by filtration and dried in vacuo.

| Intermediate | Structure | tᵣₑₜ [min] | M + H | Method |
|---|---|---|---|---|
| E-4 | 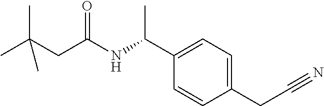 [SMILES: C[C@@H](NC(=O)CC(C)(C)C)c1ccc(CC#N)cc1] | 2.15 | 205 (M − tBu) | Method 5 |

### Synthesis of E-5

To E-4 (6.0 g, 23.0 mmol) in dry THF (80 mL), 60% sodium hydride (1.38 g, 34.5 mmol) is added at 0° C. and stirred for 30 min at this temperature. Bis(2-bromoethyl) ether (4.81 g, 20.7 mmol) is added dropwise, and the mixture is stirred for 16 h at room temperature. Water is added, and the aqueous layer is extracted exhaustively with EtOAc. The combined organic layer is dried (Na₂SO₄), filtered, and concentrated in vacuo. The crude product is purified by column chromatography (SiO₂, 10-30% EtOAc in hexane).

| Intermediate | Structure | tᵣₑₜ [min] | M + H | Method |
|---|---|---|---|---|
| E-5 | 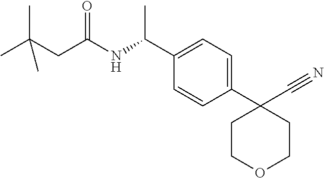 [SMILES: C[C@@H](NC(=O)CC(C)(C)C)c1ccc(C2(C#N)CCOCC2)cc1] | 2.19 | 275 (M − tBu) | Method 5 |

### Synthesis of E-6

To E-5 (3.0 g, 9.09 mmol) in EtOH (20 mL)/water (20 mL), KOH (5.08 g, 90.9 mmol) is added and stirred under reflux for 24 h. The mixture is treated with 2 N HCl, the precipitate is collected by filtration and triturated with water. The obtained solid is dried in vacuo.

| Intermediate | Structure | tᵣₑₜ [min] | M − H | Method |
|---|---|---|---|---|
| E-6 | 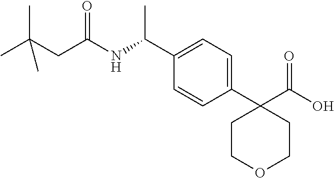 [SMILES: C[C@@H](NC(=O)CC(C)(C)C)c1ccc(C2(C(=O)O)CCOCC2)cc1] | 2.03 | 294 (M − tBu) | Method 5 |

### Synthesis of E-7

To E-6 (2.00 g, 5.73 mmol) in MeOH (30 mL), H₂SO₄ (1.52 ml) is added at 0° C. The reaction mixture is stirred under reflux for 24 h. Water is added, and the mixture is neutralized with NaHCO₃. The aqueous layer is extracted exhaustively with DCM, and the combined organic layer is dried (Na₂SO₄), filtered, and concentrated in vacuo.

| Intermediate | Structure | tᵣₑₜ [min] | M − H | Method |
|---|---|---|---|---|
| E-7 | 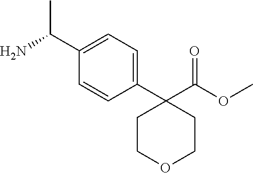 [SMILES: COC(=O)C1(c2ccc([C@@H](C)N)cc2)CCOCC1] | 1.42 | 264 | Method 5 |

### Synthesis of Examples

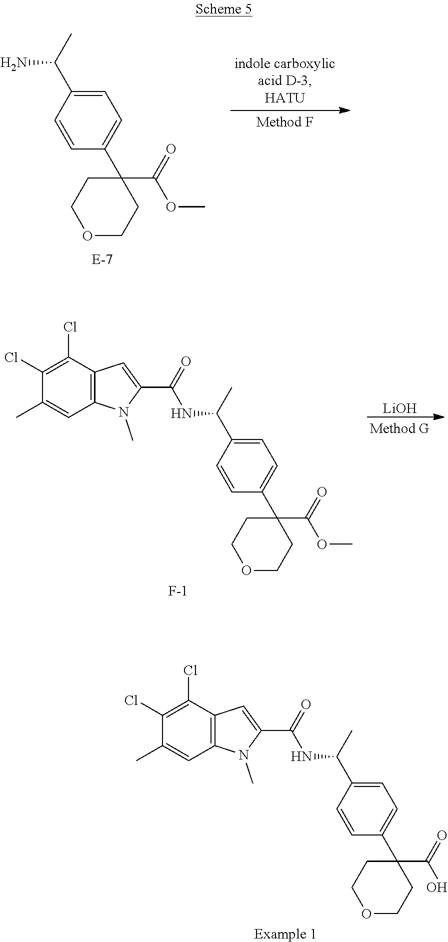

5 Synthesis of F-1 (Method F) To D-3 (8.0 g, 31.0 mmol) in dry DMF (80 mL), triethylamine (9.39 g, 93.0 mmol), HATU (11.78 g, 31.0 mmol), and HOAT (4.22 g, 31.0 mmol) are added and stirred for 5 min at 0° C. E-7 (8.98 g, 34.1 mmol) is added and stirred for 16 h at room temperature. The mixture is treated with water, the precipitate is collected by filtration and triturated with water. The crude product is dried in vacuo, triturated with diethyl ether and dried in vacuo.

The following substituted indoles are prepared in an analogous manner using the corresponding carboxylic acid intermediate of formula D.

| Intermediate | Structure | tᵣₑₜ [min] | M + H | Method |
|---|---|---|---|---|
| F-1 | 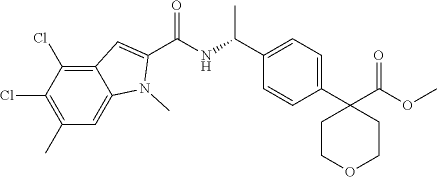 [SMILES: COC(=O)C1(c2ccc([C@@H](C)NC(=O)c3cc4c(Cl)c(Cl)c(C)cc4n3C)cc2)CCOCC1] | 2.61 | 503 | Method 5 |
| F-2 | 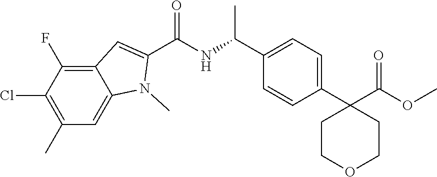 [SMILES: COC(=O)C1(c2ccc([C@@H](C)NC(=O)c3cc4c(F)c(Cl)c(C)cc4n3C)cc2)CCOCC1] | 0.84 | 487 | Method 6 |
| F-3 | 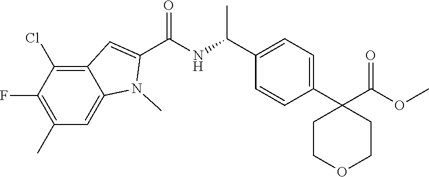 [SMILES: COC(=O)C1(c2ccc([C@@H](C)NC(=O)c3cc4c(Cl)c(F)c(C)cc4n3C)cc2)CCOCC1] | 0.83 | 487 | Method 6 |
| F-4 | 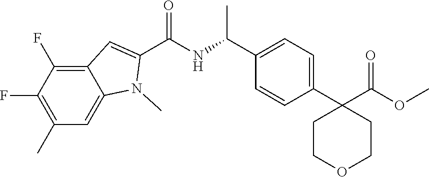 [SMILES: COC(=O)C1(c2ccc([C@@H](C)NC(=O)c3cc4c(F)c(F)c(C)cc4n3C)cc2)CCOCC1] | 0.79 | 471 | Method 6 |
| F-5 | 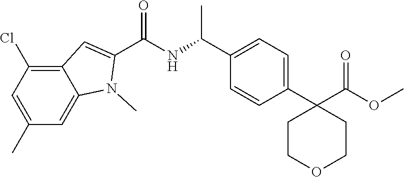 [SMILES: COC(=O)C1(c2ccc([C@@H](C)NC(=O)c3cc4c(Cl)cc(C)cc4n3C)cc2)CCOCC1] | 0.82 | 469 | Method 6 |
| F-6 | 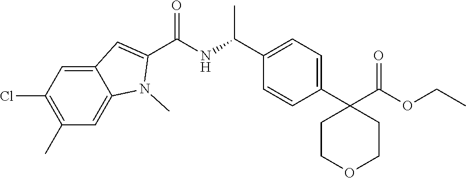 [SMILES: CCOC(=O)C1(c2ccc([C@@H](C)NC(=O)c3cc4cc(Cl)c(C)cc4n3C)cc2)CCOCC1] | 0.86 | 483 | Method 6 |

### Synthesis of Example 1 (Method G)

To F-1 (8.20 g, 16.3 mmol) in MeOH (32 mL)/THF (32 mL)/water (32 mL), LiOH monohydrate (6.84 g, 162.6 mmol) is added and stirred for 48 h at room temperature. The mixture is treated with 4 N HCl, the precipitate is collected by filtration and triturated with water. The crude product is purified by prep. HPLC-MS.

| Intermediate | Structure | tᵣₑₜ [min] | M + H | Method |
|---|---|---|---|---|
| 1 | 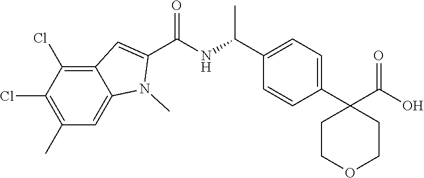 [SMILES: Cc1cc2c(cc(C(=O)N[C@H](C)c3ccc(C4(C(=O)O)CCOCC4)cc3)n2C)c(Cl)c1Cl] | 1.15 | 489 | Method 4 |
| 2 | 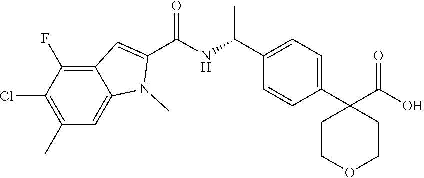 [SMILES: Cc1cc2c(cc(C(=O)N[C@H](C)c3ccc(C4(C(=O)O)CCOCC4)cc3)n2C)c(F)c1Cl] | 1.10 | 473 | Method 4 |
| 3 | 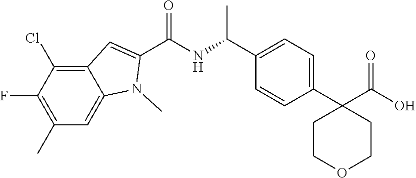 [SMILES: Cc1cc2c(cc(C(=O)N[C@H](C)c3ccc(C4(C(=O)O)CCOCC4)cc3)n2C)c(Cl)c1F] | 1.08 | 473 | Method 4 |
| 4 | 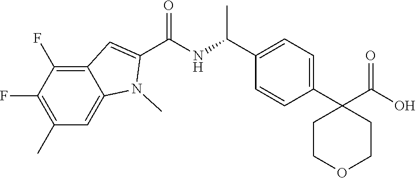 [SMILES: Cc1cc2c(cc(C(=O)N[C@H](C)c3ccc(C4(C(=O)O)CCOCC4)cc3)n2C)c(F)c1F] | 1.05 | 457 | Method 4 |
| 5 | 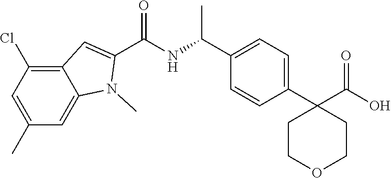 [SMILES: Cc1cc(Cl)c2cc(C(=O)N[C@H](C)c3ccc(C4(C(=O)O)CCOCC4)cc3)n(C)c2c1] | 1.05 | 455 | Method 4 |
| 6 | 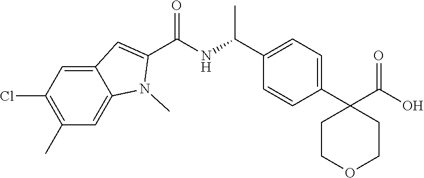 [SMILES: Cc1cc2c(cc1Cl)cc(C(=O)N[C@H](C)c1ccc(C3(C(=O)O)CCOCC3)cc1)n2C] | 1.08 | 455 | Method 4 |

### Biological Examples

The invention will now be illustrated with reference to the following non-limiting Examples describing biological activity and properties of the compounds of the invention.

3-Phosphoglycerate dehydrogenase (PHGDH) Fluorescence Intensity Assay This assay is used to identify compounds which inhibit the enzymatic activity of PHGDH which catalyzes the reaction of 3-Phosphoglycerate (3-PG) and NAD to 3-Phosphohydroxypyruvate and NADH.

The produced NADH is used in a coupled reaction for Diaphorase mediated reduction of Resazurin to Resorufin which can be measured in a Fluorescence Intensity readout.

The full length version of PHGDH enzyme was expressed in BL21 (DE3) E. coli through transformation with a plasmid containing the PHGDH cDNA with an N-terminal HIS-tag and a TEV cleavage site. The recombinant protein was then isolated with Ni-NTA beads and eluted on MONO Q ion exchange chromatography columns. The fractions corresponding to PHGDH were de-salted and concentrated to be used in the biochemical assay.

The 3-phosphoglycerate substrate was purchased from Sigma. NAD, Diaphorase and Resazurin were purchased from Sigma Aldrich.

Compounds are dispensed onto assay plates (black, low volume, flat bottom 384 well, Corning) using an Access Labcyte Workstation with the Labcyte Echo 55× from a DMSO solution. For the chosen highest assay concentration of 100 μM, 150 nl of compound solution are transferred from a 10 mM DMSO compound stock solution. A series of 11 concentrations (10 1:5 steps) is transferred for each compound.

DMSO is added such that every well has a total of 150 nl compound solution.

The assay has been performed using 500 μM NAD and 500 μM 3-PG (final assay concentrations).

5 μl of PHGDH protein (final assay concentration 100 ng/ml) in assay buffer (125 mM Tris-HCl, pH 7.5; 56.25 mM hydrazine sulfate pH 9.0; 2.5 mM EDTA; assay specific NAD concentration; 0.0125% Tween20) are added to the 150 nl of compounds.

10 μl of a mix containing assay specific 3-PG concentration (500 μM), Resazurin (25 UM final assay concentration) and Diaphorase (35 μg/ml final assay concentration) are added. Plates are kept at room temperature. After 240 minutes incubation time the fluorescence signal is measured in a PerkinElmer Envision HTS Multilabel Reader with an excitation wavelength at 530-560 nm and an emission wavelength at 590 nm.

Each plate contains negative controls (diluted DMSO instead of test compound; reaction as described with PHGDH protein) and positive controls (diluted DMSO instead of test compound; reaction as described with buffer instead of PHGDH protein). Negative and positive control values are used for normalization.

A known inhibitor of PHGDH activity is used as internal control.

IC₅₀ values are calculated and analyzed in the MEGALAB IC₅₀ application using a 4 parametric logistic model.

TABLE 1

| Example | IC₅₀ PHGDH |
|---|---|
| 1 | 6 nM |
| 2 | 27 nM |
| 3 | 10 nM |
| 4 | 31 nM |
| 5 | 25 nM |
| 6 | 943 nM |

### Drug Transport Across Human Caco-2 Cells for the Estimation of Intestinal Drug Absorption in Man

The assay provides information on the potential of a compound to pass the cell membrane, on the extent of oral absorption as well as on whether the compound is actively transported by uptake and/or efflux transporters. Permeability measurements across polarized, confluent Caco-2 cell monolayers grown on permeable filter supports are used as the in vitro absorption model. Apparent permeability coefficients (PE) of the compounds across the Caco-2 monolayers are measured (pH 7.2, 37° C.) in apical-to-basal (AB) (absorptive) and basal-to-apical (BA) (secretory) transport direction. AB permeability (PEAB) represents drug absorption from the intestine into the blood and BA permeability (PEBA) drug secretion from the blood back into the intestine via both passive permeability as well as active transport mechanisms mediated by efflux and uptake transporters that are expressed on the Caco-2 cells. The compounds are assigned to permeability/absorption classes by comparison of the AB permeabilities with the AB permeabilities of reference compounds with known in vitro permeability and oral absorption in the human. Identical or similar permeabilities in both transport directions indicate passive permeation, vectorial permeability points to additional active transport mechanisms. Higher PEBA than PEAB suggests the involvement of an apical efflux transporter (like P-gp) and/or basolateral uptake transporter; higher PEAB than PEBA permeability suggests involvement of an apical uptake transporter (like PepT1) and/or basolateral efflux transporter (like MRP3). Active transport is concentration-dependently saturable.

Caco-2 cells obtained from the Leibniz Institute DSMZ-German Collection of Microorganisms and Cell Cultures GmbH (1-2×10⁵ cells/1 cm² area) are seeded on filter inserts (Costar transwell polycarbonate or PET filters, 0.4 μm pore size) and cultured (DMEM) for 10 to 25 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO₄, 1.8 mM CaCl₂), 4.17 mM NaHCO₃, 1.19 mM Na₂HPO₄×7H₂O, 0.41 mM NaH₂PO₄×H₂O, 15 mM HEPES, 20 mM glucose, pH 7.2) containing 0.25% BSA to prepare the transport solutions (0.1-300 μM compound, final DMSO≤0.5%). The transport solution (TL) is applied to the apical or basolateral donor side for measuring A-B or B-A permeability (3 filter replicates), respectively. The receiver side contains HTP-4 buffer supplemented with 0.25% BSA. Samples are collected at the start and end of experiment from the donor and at various time intervals (0, 30, 60, and 90 minutes) for up to 2 hours also from the receiver side for concentration measurement by HPLC-MS/MS or scintillation counting. Sampled receiver volumes are replaced with fresh receiver solution. Compounds are assigned to the following permeability/oral absorption classes:
Very low: 1×10⁻⁷ cm/sec<PEAB
Low: 1×10⁻⁷ cm/s<PEAB<6×10⁻⁷ cm/sec
Moderate: 6×10⁻⁷ cm/sec<PEAB<5×10⁻⁶ cm/sec
Good: 5×10⁻⁶ cm/s<PEAB<1×10⁻⁵ cm/sec
Very good: PEAB<1×10⁻⁵ cm/sec

Compounds are classified with respect to (permeability) efflux ratios (PEBA/PEAB) given below whether they are likely to be actively transported:
no: 0.67<=ratio <=1.5
probable (active efflux): 1.5<ratio <=2
probable (active uptake): 0.67<ratio <=0.5
yes (active efflux): 2<ratio
yes (active uptake): ratio <0.5

TABLE 2

| | PEAB (×10⁻⁶ | PEBA (×10⁻⁶ | efflux |
|---|---|---|---|
| Example | cm/sec) | cm/sec) | ratio |
| 1 | 17.1 | 14.6 | 0.9 |
| 2 | 11.0 | 13.0 | 1.2 |
| 3 | 16.0 | 9.1 | 0.6 |
| 4 | 12.0 | 9.2 | 0.8 |
| 5 | 10.0 | 12.0 | 1.2 |
| 6 | 7.9 | 8.4 | 1.1 |

TABLE 3

| | PEAB | PEBA | |
|---|---|---|---|
| | (×10⁻⁶ | (×10⁻⁶ | efflux |
| Comparative Example | cm/sec) | cm/sec) | ratio |
| 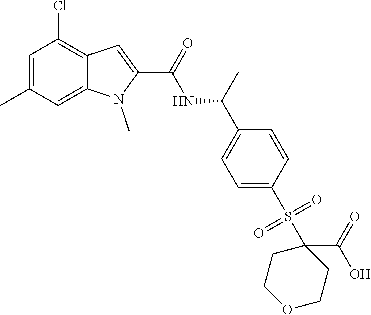 [SMILES: Cc1cc(Cl)c2cc(C(=O)N[C@H](C)c3ccc(S(=O)(=O)C4(C(=O)O)CCOCC4)cc3)n(C)c2c1] | <0.4 | 0.2 | |
| Comp-1 | | | |
| (07-002 in WO2018/167019) | | | |
| 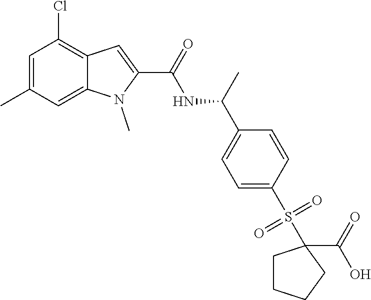 [SMILES: Cc1cc(Cl)c2cc(C(=O)N[C@H](C)c3ccc(S(=O)(=O)C4(C(=O)O)CCCC4)cc3)n(C)c2c1] | 0.2 | 0.4 | 1.8 |
| Comp-2 | | | |
| (07-004 in WO2018/167019) | | | |
| 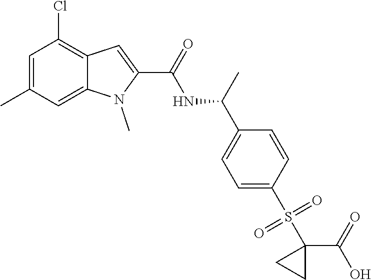 [SMILES: Cc1cc(Cl)c2cc(C(=O)N[C@H](C)c3ccc(S(=O)(=O)C4(C(=O)O)CC4)cc3)n(C)c2c1] | 0.1 | 1.7 | 17.0 |
| Comp-3 | | | |
| (07-013 in WO2018/167019) | | | |

The formulation examples which follow illustrate the present invention without restricting its scope:

### Examples of Pharmaceutical Formulations

A)

| Tablets | per tablet | |
|---|---|---|
| active substance according to formula (I) | 100 | mg |
| lactose | 140 | mg |
| corn starch | 240 | mg |
| polyvinylpyrrolidone | 15 | mg |
| magnesium stearate | 5 | mg |
| | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B)

| Tablets | per tablet | |
|---|---|---|
| active substance according to formula (I) | 80 | mg |
| lactose | 55 | mg |
| corn starch | 190 | mg |
| microcrystalline cellulose | 35 | mg |
| polyvinylpyrrolidone | 15 | mg |
| sodiumcarboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium carboxymethyl starch and the magnesium stearate are added and mixed in and the mixture is compressed to form tablets of a suitable size.

C)

| Tablets | per tablet | |
|---|---|---|
| active substance according to formula (I) | 25 | mg |
| lactose | 50 | mg |
| microcrystalline cellulose | 24 | mg |
| magnesium stearate | 1 | mg |
| | 100 | mg |

The active substance, lactose and cellulose are mixed together. The mixture is screened, then either moistened with water, kneaded, wet-granulated and dried or dry-granulated or directly final blend with the magnesium stearate and compressed to tablets of suitable shape and size. When wet-granulated, additional lactose or cellulose and magnesium stearate is added and the mixture is compressed to produce tablets of suitable shape and size.

D)

| Ampoule solution | |
|---|---|
| active substance according to formula (I) | 50 mg |
| sodium chloride | 50 mg |
| water for inj. | 5 mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of formula (I): 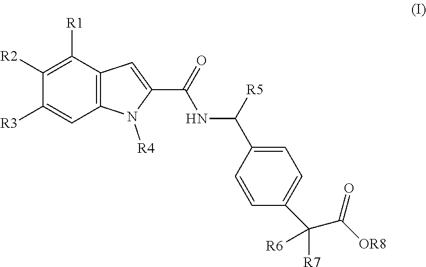
wherein:
R1, R2 and R3 are each independently selected from the group consisting of: hydrogen, C₁₋₃alkyl and halogen;
R4 is C₁₋₄alkyl;
R5 is C₁₋₃alkyl or hydroxy-C₁₋₃alkyl;
R6 and R7 together form a heterocyclyl ring containing one or more moieties selected from the group consisting of: —O—, —N(H)—, —N(COR^{a})—, —N(SO₂R^{a})—, —S—, —S(O)— and —S(O)₂—, wherein said heterocyclyl ring is optionally substituted by one or more substituents, each independently selected from —C(O)C₁₋₃alkyl and —N(H)COR^{a};
R8 is hydrogen or C₁₋₃alkyl;
R^{a} is selected from the group consisting of: C₁₋₃alkyl, —NH₂, —N(H)C₁₋₃alkyl and —N(C₁₋₃alkyl)₂;
or a pharmaceutically acceptable salt thereof.

2. The compound or the pharmaceutically acceptable salt according to claim 1,
wherein R1 is halogen and/or R3 is C₁₋₃alkyl.

3. The compound or the pharmaceutically acceptable salt according to claim 1, wherein R2 is hydrogen or halogen.

4. The compound or the pharmaceutically acceptable salt according to claim 1, wherein at least one of R1, R2 and/or R3 is halogen.

5. The compound or the pharmaceutically acceptable salt according to claim 1, wherein R3 is methyl.

6. The compound or the pharmaceutically acceptable salt according to claim 1, wherein R4 is methyl.

7. The compound or the pharmaceutically acceptable salt according to claim 1, wherein R5 is methyl.

8. The compound or the pharmaceutically acceptable salt according to claim 1, wherein R6 and R7 together form a 3-7 membered saturated heterocyclyl ring containing a moiety selected from the group consisting of: —O—, —N(H)— and —S—.

9. The compound or the pharmaceutically acceptable salt according to according to claim 1, wherein R6 and R7 together form a tetrahydropyrane ring.

10. The compound or the pharmaceutically acceptable salt according to claim 1, wherein R6 and R7 together form:

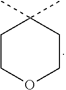

11. The compound or the pharmaceutically acceptable salt according to claim 1, wherein R8 is hydrogen.

12. The compound or the pharmaceutically acceptable salt according to claim 1, being selected from:

| Example | Structure |
|---|---|
| 1 | 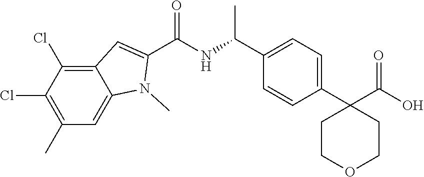 [SMILES: Cc1cc2c(cc(C(=O)N[C@H](C)c3ccc(C4(C(=O)O)CCOCC4)cc3)n2C)c(Cl)c1Cl] |
| 2 | 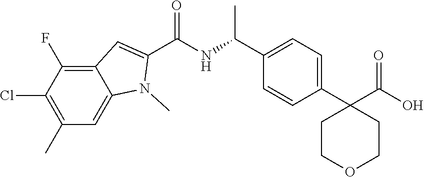 [SMILES: Cc1cc2c(cc(C(=O)N[C@H](C)c3ccc(C4(C(=O)O)CCOCC4)cc3)n2C)c(F)c1Cl] |
| 3 | 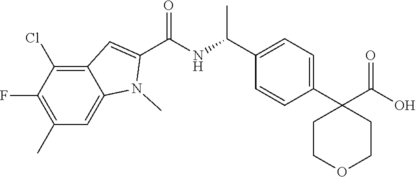 [SMILES: Cc1cc2c(cc(C(=O)N[C@H](C)c3ccc(C4(C(=O)O)CCOCC4)cc3)n2C)c(Cl)c1F] |
| 4 | 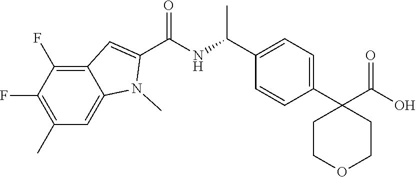 [SMILES: Cc1cc2c(cc(C(=O)N[C@H](C)c3ccc(C4(C(=O)O)CCOCC4)cc3)n2C)c(F)c1F] |
| 5 | 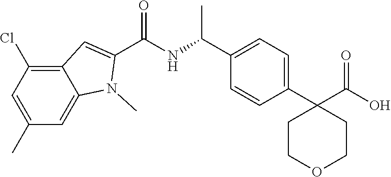 [SMILES: Cc1cc(Cl)c2cc(C(=O)N[C@H](C)c3ccc(C4(C(=O)O)CCOCC4)cc3)n(C)c2c1] |
| 6 | 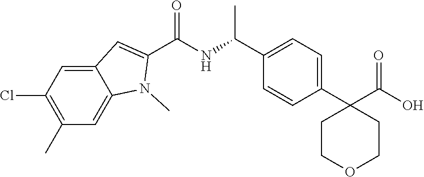 [SMILES: Cc1cc2c(cc1Cl)cc(C(=O)N[C@H](C)c1ccc(C3(C(=O)O)CCOCC3)cc1)n2C] |

or a pharmaceutically acceptable salt thereof.

13. The compound or the pharmaceutically acceptable salt as defined in claim 1 for use as a medicament.

14. A method of treatment and/or prevention of cancer, infections, inflammations and/or autoimmune diseases comprising the administration to a patient in need thereof of the compound or the pharmaceutically acceptable salt as defined in claim 1.

15. A pharmaceutical composition comprising the compound or the pharmaceutically acceptable salt as defined of claim 1 and a pharmaceutically acceptable excipient and optionally one or more further pharmacologically active substance(s).
